(19) United States
(12) Plant Patent Application Publication
(10) Pub. No.: US 2003/0121085 P1
(43) Pub. Date: Jun. 26, 2003

(54) Apple tree named 'Nicogreen'

(76) Inventor: Johan Nicolai, Sint-Truiden-Gorsem (BE)

Correspondence Address: STRATTON BALLEW, 213 S 12TH AVE, YAKIMA, WA, 98902, US

(21) Appl. No.: 10/293,208

(22) Filed: Nov. 12, 2002

Related U.S. Application Data
(60) Provisional application No. 60/338,027, filed on Nov. 13, 2001.

(51) Int. Cl. A01H 5/00

(52) U.S. Cl. PLT/172

(57) ABSTRACT

A new apple tree is disclosed. The new variety is the result of a cross between ‘Delcorf’ and ‘Granny Smith,’ and is most similar in appearance to ‘Granny Smith.’ The new variety is distinguishable from ‘Granny Smith’ by its early maturation, which occurs approximately six weeks before ‘Granny Smith.’

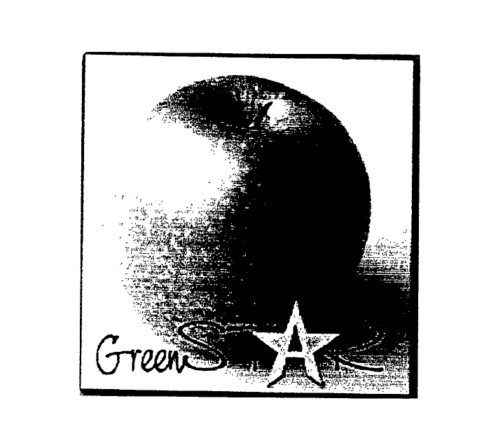

### PRIORITY CLAIM

This application claims priority under U.S. Provisional Application No. 60/338,027 filed Nov. 13, 2002.

### FIELD OF THE INVENTION

The invention relates to the field of fruit trees. More specifically, the invention consists of a new and distinct variety of apple tree (Malus domestica).

### ORIGIN OF THE VARIETY

The new variety originated as a controlled cross between Delcorf (unpatented) and Granny Smith (unpatented).

### ASEXUAL REPRODUCTION OF THE VARIETY

The new variety has been asexually propagated, and has been demonstrated to remain true to type through successive generations.

### BRIEF DESCRIPTION OF THE DRAWING

The FIGURE illustrates a representative fruit of the new variety.

### DETAILED DESCRIPTION OF THE VARIETY

The following is a detailed botanical description of the new and distinct variety of apple tree, based on observations of specimens grown in Belgium.

The new variety most closely resembles ‘Granny Smith’. However, fruit of the new variety matures approximately six weeks earlier than ‘Granny Smith.’

Tree: Vigor Medium (compare to ‘Golden Delicious’)

Fruit: Shape Oblong (compare to ‘Gravenstein’)

Over color of skin.—Green (compare to ‘Granny Smith’).

Time of beginning of flowering (10% open flowers): Medium (compare to ‘Cox's Orange Pippin’)

Time of fruit ripening for eating: Medium (compare to ‘Reine des Reinettes’)

Sensitivity to pests/disease: Not very sensitive

Sensitivity to scald: Not very sensitive

I claim:

1. A new and distinct variety of apple tree as described and claimed herein.
